(19) United States
(12) Patent Application Publication
(10) Pub. No.: US 2007/0167502 A1
(43) Pub. Date: Jul. 19, 2007

(54) Association between a heterocyclic compound and an antioxidant agent

(76) Inventors: Louis Casteilla, Escalquens (FR); Luc Penicaud, Toulouse (FR); Pascal Berthelot, Haubourdin (FR); Catherine Dacquet, Paris (FR); Daniel-Henri Caignard, Boisemont (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/593,681

(22) PCT Filed: Mar. 30, 2005
(86) PCT No.: PCT/FR05/00755

§ 371 Date: Sep. 20, 2006

(30) Foreign Application Priority Data
Mar. 31, 2004 (FR) 04.03359

(51) Int. Cl. A61K 31/427; A61K 31/423; A61K 31/404; A61K 31/355; A61K 31/12

(52) U.S. Cl. 514/367; 514/375; 514/415; 514/458; 514/690

(57) ABSTRACT

A pharmaceutical composition for treatment of obesity associated with lipid and carbohydrate metabolism comprising (i) a substance which is useful in promoting lipid and carbohydrate metabolism, (ii) an antioxidant agent and, optionally, (iii) a pharmaceutically acceptable carrier or excipient, wherein the substance which promotes lipid and carbohydrate metabolism and the antioxidant agent are present in therapeutically effective dosages. Methods which are useful in treating conditions associated with obesity.

The present invention relates to a new association between a heterocyclic compound and an antioxidant agent for obtaining pharmaceutical compositions for use in the treatment and/or prevention of obesity and of overweight characterised by a body mass index greater than 25.

Obesity is a major public health problem in all developed countries. It is also increasing steadily in developing countries and is affecting an ever younger population. Obesity is a well-established risk factor for cardiovascular diseases and is associated with a significantly increased risk of cerebro-vascular accidents, non-insulin-dependent diabetes, vesicular calculi, respiratory dysfunction, osteoarthritis, several forms of cancer and premature death.

It has been found that, in obese people, the generation of reactive oxygenated species released by monocytes and leukocytes is greatly increased with respect to non-obese subjects (J. Clin. Endocrinol. Metab., 2001, 86, 355-362). Elevated plasma concentrations of alpha tumour necrosis factor (TNFα) in obese people stimulate inflammatory processes (J. Clin. Endocrinol. Metab., 1998, 83, 2907-2910) and are responsible for the generation of reactive oxygenated species by leukocytes (Oncogene, 1998, 17, 1639-1651). The pathological state of obesity is also associated with increased oxidation of lipids and proteins, which may be the cause of high plasma levels of 9- and 13-hydroxy-octadecadienoic acids (9-HODE and 13-HODE) (Totowa: Humano. Press., 1998, 147-155), key indices of lipid peroxidation (J. Clin. Endocrinol. Metab., 2001, 86, 355-362). In parallel, the “antioxidant” capabilities of the body are reduced.

In obese subjects, it has been shown that excessive food intake causes major lipid and protein damage. Over-consumption of calories by obese people can cause the formation of free radicals and expose them to significant oxidative lesions which help to maintain the state of obesity.

The specific markers of oxidation are significantly reduced by a 48-hour fast or by calorie restriction accompanying weight loss (J. Clin. Endocrinol. Metab., 2001, 86, 355-362).

A strategy aimed at reducing the “oxidative burden” on the body by favouring the lipid and carbohydrate metabolisms should result in an exacerbation of the effects and, as a consequence, in weight loss in obese or overweight subjects.

The present invention relates more specifically to the association between a compound favouring the lipid and carbohydrate metabolisms of the body and an antioxidant agent.

This association is novel and exhibits pharmacological properties that are entirely surprising in the area of obesity.

More specifically, the invention relates to the association between a compound favouring the lipid and carbohydrate metabolisms which has a heterocyclic structure, and an antioxidant agent.

The heterocyclic compounds favouring the lipid and carbohydrate metabolisms in accordance with the invention are, more specifically, compounds of formula (I): 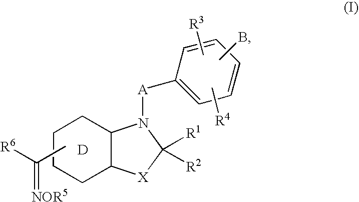
wherein:
X represents an oxygen or sulphur atom, or a group CH₂ or 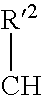
(wherein R′² together with R² forms an additional bond),
R¹ and R², which may be the same or different, each represent a hydrogen atom, a linear or branched (C₁-C₆)alkyl group, an aryl group, an aryl-(C₁-C₆)alkyl group in which the alkyl moiety is linear or branched, an aryloxy group, an aryl-(C₁-C₆)alkyloxy group in which the alkyl moiety is linear or branched, a linear or branched (C₁-C₆)alkoxy group, a hydroxy group, an amino group, a linear or branched (C₁-C₆)alkylamino group or a di-(C₁-C₆)alkylamino group in which the alkyl moieties are linear or branched,
   or R¹ and R² together form an oxo, thioxo or imino group, it also being possible for R² together with R′² to form an additional bond,
A represents a (C₁-C₆)alkylene chain in which one CH₂ group may be replaced by a hetero atom selected from oxygen and sulphur or by a group NRₐ (wherein Rₐ represents a hydrogen atom or a linear or branched (C₁-C₆)alkyl group), or by a phenylene or naphthylene group,
R³ and R⁴, which may be the same or different, each represent a hydrogen or halogen atom or a group R, OR or NRR′ (wherein R and R′, which may be the same or different, each represent a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety is linear or branched, an aryl-(C₂-C₆)alkenyl group in which the alkenyl moiety is linear or branched, an aryl-(C₂-C₆)alkynyl group in which the alkynyl moiety is linear or branched, a heteroaryl group, a heteroaryl-(C₁-C₆)alkyl group in which the alkyl moiety is linear or branched, a heteroaryl-(C₂-C₆)alkenyl group in which the alkenyl moiety is linear or branched, a heteroaryl-(C₂-C₆)alkynyl group in which the alkynyl moiety is linear or branched, a (C₃-C₈)cycloalkyl group, a (C₃-C₈)cycloalkyl-(C₁-C₆)alkyl group in which the alkyl moiety is linear or branched, or a linear or branched (C₁-C₆)polyhaloalkyl group),
   or R³ and R⁴, together with the carbon atoms carrying them, when they are carried by two adjacent carbon atoms, form a ring that has 5 or 6 ring members and that may contain a hetero atom selected from oxygen, sulphur and nitrogen,
R⁵ and R⁶, which may be the same or different, may have any of the meanings of R given hereinbefore,
D represents:
   a benzene nucleus, in which case X cannot represent a group 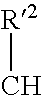
   as defined hereinbefore,
   or D represents a pyridine, pyrazine, pyrimidine or pyridazine nucleus,
B represents a linear or branched (C₁-C₆)alkyl group or a linear or branched (C₂-C₆)-alkenyl group, those groups being substituted:
   by a group of formula (II): 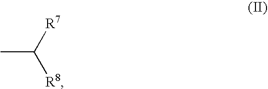
   wherein:
   R⁷ represents a group 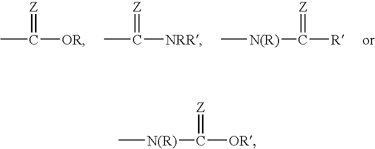
   wherein Z represents an oxygen or sulphur atom, and R and R′, which may be the same or different, may have any of the meanings given hereinbefore,
   and R⁸ represents an aryl group, an arylalkyl group wherein the alkyl moiety contains from 1 to 6 carbon atoms and may be linear or branched, a heteroaryl group, a heteroarylalkyl group wherein the alkyl moiety contains from 1 to 6 carbon atoms and may be linear or branched, CN, tetrazole, 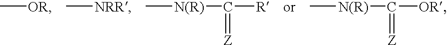
   wherein Z is as defined hereinbefore, and R and R′, which may be the same or different, may have any of the meanings given hereinbefore,
   or by a group R⁹, wherein R⁹ represents a CN, tetrazole, 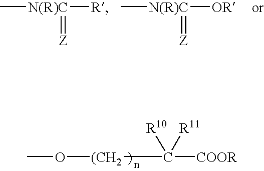
   group, wherein Z is as defined hereinbefore, and R and R′, which may be the same or different, may have any of the meanings given hereinbefore, n represents 0, 1, 2, 3, 4, 5 or 6, and R¹⁰ and R¹¹, which may be the same or different, each represent a hydrogen atom or a linear or branched (C₁-C₆)alkyl group, it being understood that R¹⁰ and R¹¹ cannot simultaneously represent a hydrogen atom,
or B represents a group of formula (II) or a group R⁹ as defined hereinbefore,
it being understood that:
the oxime R⁶—C(═N—OR⁵)- can be of Z or E configuration,
aryl means a phenyl, naphthyl or biphenyl group, it being possible for those groups to be partially hydrogenated,
heteroaryl means any mono- or bi-cyclic aromatic group containing 5 to 10 members, which may be partially hydrogenated in one of the rings in the case of bicyclic heteroaryls and which contains 1 to 3 hetero atoms selected from oxygen, nitrogen and sulphur,
it being possible for the aryl and heteroaryl groups thereby defined to be substituted by from 1 to 3 groups selected from linear or branched (C₁-C₆)alkyl, linear or branched (C₁-C₆)polyhaloalkyl, linear or branched (C₁-C₆)alkoxy, hydroxy, carboxy, formyl, NR_{b}R_{c} (wherein R_{b} and R_{c}, which may be the same or different, each represent a hydrogen atom, a linear or branched (C₁-C₆)alkyl group, an aryl group or a heteroaryl group), ester, amido, nitro, cyano, and halogen atoms,
their enantiomers and diastereoisomers, and also addition salts thereof with a pharmaceutically acceptable acid or base.
Advantageously, the heterocyclic compounds of the association according to the invention are compounds of formula (I) wherein:
X represents a sulphur atom,
R¹ and R² together form an oxo group,
A represents a chain —CH₂—CH₂—O—,
R³ and R⁴ simultaneously represent a hydrogen atom,
R⁵ represents a hydrogen atom or an alkyl group.
R⁶ represents a phenyl or substituted phenyl group, more especially substituted by a halogen atom,
D represents a benzene nucleus,
B represents a group 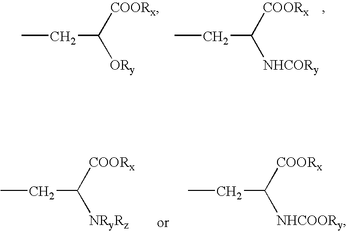
wherein Rₓ, R_{y}and R_{z}, which may be the same or different, each represent:
a hydrogen atom or an alkyl group such as, for example, a methyl, ethyl, propyl, isopropyl, butyl, isobutyl, tert-butyl, pentyl, isopentyl, neopentyl or hexyl group,
a polyhaloalkyl group such as, for example, a trifluoromethyl or trifluoroethyl group, or a phenyl or benzyl group.

Even more preferably, the heterocyclic compounds of the association according to the invention are:
methyl 2-ethoxy-3-{4-[2-(6-[(E)(methoxyimino)(phenyl)methyl]-2-oxo-1,3-benzothiazol-3(2H)-yl)ethoxy]phenyl}propanoate,
methyl 2-ethoxy-3-{4-[2-(6-[(Z)(hydroxyimino)(phenyl)methyl]-2-oxo-1,3-benzothiazol-3(2H)-yl)ethoxy]phenyl}propanoate,
methyl 3-{4-[2-(6-[(methoxyimino)(phenyl)methyl]-2-oxo-1,3-benzothiazol-3(2H)-yl)ethoxy]phenyl}-2-(2,2,2-trifluoroethoxy)propanoate,
methyl 3-{4-[2-(6-[(hydroxyimino)(phenyl)methyl]-2-oxo-1,3-benzothiazol-3(2H)-yl)ethoxy]phenyl}-2-(2,2,2-trifluoro-ethoxy)propanoate,
2-ethoxy-3-{4-[2-(6-[(methoxyimino)(phenyl)methyl]-2-oxo-1,3-benzothiazol-3(2 H)-yl)ethoxy]phenyl}propanoic acid,
2-ethoxy-3-{4-[2-(6-[(E)(methoxyimino)(phenyl)methyl]-2-oxo-1,3-benzothiazol-3(2H)-yl)ethoxy]phenyl}propanoic acid,
2-ethoxy-3-{4-[2-(6-[(E)(methoxyimino)(phenyl)methyl]-2-oxo-1,3-benzothiazol-3(2H)-yl)ethoxy]phenyl}propanoic acid, enantiomer 1,
2-ethoxy-3-{4-[2-(6-[(E)(methoxyimino)(phenyl)methyl]-2-oxo-1,3-benzothiazol-3(2H)-yl)ethoxy]phenyl}propanoic acid, enantiomer 2,
2-ethoxy-3-{4-[2-(6-[(Z)(methoxyimino)(phenyl)methyl]-2-oxo-1,3-benzothiazol-3(2H)-yl)ethoxy]phenyl}propanoic acid,
2-ethoxy-3-{4-[2-(6-[(Z)(methoxyimino)(phenyl)methyl]-2-oxo-1,3-benzothiazol-3(2H)-yl)ethoxy]phenyl}propanoic acid, enantiomer 1,
2-ethoxy-3-{4-[2-(6-[(Z)(methoxyimino)(phenyl)methyl]-2-oxo-1,3-benzothiazol-3(2H)-yl)ethoxy]phenyl}propanoic acid, enantiomer 2,
2-ethoxy-3-{4-[2-(6-[(hydroxyimino)(phenyl)methyl]-2-oxo-1,3-benzothiazol-3(2H)-yl)ethoxy]phenyl}propanoic acid,
2-ethoxy-3-{4-[2-(6-[(E)(hydroxyimino)(phenyl)methyl]-2-oxo-1,3-benzothiazol-3(2h)-yl)ethoxy]phenyl}propanoic acid,
2-ethoxy-3-{4-[2-(6-[(Z)(hydroxyimino)(phenyl)methyl]-2-oxo-1,3-benzothiazol-3(2H)-yl)ethoxy]phenyl}propanoic acid,
methyl 3-{4-[2-(6-[(Z)(3-chlorophenyl)(methoxyimino)methyl]-2-oxo-1,3-benzothiazol-3(2H)-yl)ethoxy]phenyl}-2-ethoxypropanoate,
methyl 3-{4-[2-(6-[(3-chlorophenyl)(hydroxyimino)methyl]-2-oxo-1,3-benzothiazol-3(2H)-yl)ethoxy]phenyl}-2-ethoxypropanoate,
methyl 2-methoxy-3-{4-[2-(6-[(methoxyimino)(phenyl)methyl]-2-oxo-1,3-benzothiazol-3(2H)-yl)ethoxy]phenyl}propanoate,
3-{4-[2-(6-[(Z)(3-chlorophenyl)(methoxyimino)methyl]-2-oxo-1,3-benzothiazol-3(2H)-yl)ethoxy]phenyl}-2-ethoxypropanoic acid,
3-{4-[2-(6-[(methoxyimino)(phenyl)methyl]-2-oxo-1,3-benzothiazol-3(2H)-yl)ethoxy]phenyl}-2-(2,2,2-trifluoroethoxy)propanoic acid,
3-{4-[2-(6-[(E)(methoxyimino)(phenyl)methyl]-2-oxo-1,3-benzothiazol-3(2H)-yl)ethoxy]phenyl}-2-(2,2,2-trifluoroethoxy)propanoic acid,
3-{4-[2-(6-[(E)(methoxyimino)(phenyl)methyl]-2-oxo-1,3-benzothiazol-3(2H)-yl)ethoxy]phenyl}-2-(2,2,2-trifluoroethoxy)propanoic acid, enantiomer 1,
3-{4-[2-(6-[(E)(methoxyimino)(phenyl)methyl]-2-oxo-1,3-benzothiazol-3(2H)-yl)ethoxy]phenyl}-2-(2,2,2-trifluoroethoxy)propanoic acid, enantiomer 2,
3-{4-[2-(6-[(Z)(methoxyimino)(phenyl)methyl]-2-oxo-1,3-benzothiazol-3(2H)-yl)ethoxy]phenyl}-2-(2,2,2-trifluoroethoxy)propanoic acid,
3-{4-[2-(6-[(Z)(methoxyimino)(phenyl)methyl]-2-oxo-1,3-benzothiazol-3(2H)-yl)ethoxy]phenyl}-2-(2,2,2-trifluoroethoxy)propanoic acid, enantiomer 1,
3-{4-[2-(6-[(Z)(methoxyimino)(phenyl)methyl]-2-oxo-1,3-benzothiazol-3(2H)-yl)ethoxy]phenyl}-2-(2,2,2-trifluoroethoxy)propanoic acid, enantiomer 2,
3-{4-[2-(6-[(tert-butoxyimino)(phenyl)methyl]-2-oxo-1,3-benzothiazol-3(2H)-yl)ethoxy]phenyl}-2-ethoxypropanoic acid,
methyl 2-[(tert-butoxycarbonyl)amino]-3-{4-[2-(6-[(methoxyimino)(phenyl)methyl]-2-oxo-1,3-benzothiazol-3(2H)-yl)ethoxy]phenyl}propanoate,
methyl 2-[(tert-butoxycarbonyl)amino]-3-{4-[2-(6-[(hydroxyimino)(phenyl)methyl]-2-oxo-1,3-benzothiazol-3(2H)-yl)ethoxy]phenyl}propanoate,
methyl 2-[(butoxycarbonyl)amino]-3-{4-[2-(6-[(methoxyimino)(phenyl)methyl]-2-oxo-1,3-benzothiazol-3(2H)-yl)ethoxy]phenyl}propanoate,
methyl 3-{4-[2-(6-[(methoxyimino)(phenyl)methyl]-2-oxo-1,3-benzothiazol-3(2H)-yl)ethoxy]phenyl}-2-[(phenoxycarbonyl)amino]propanoate,
methyl 2-{[(benzyloxy)carbonyl]amino}-3-{4-[2-(6-[(hydroxyimino)(phenyl)methyl]-2-oxo-1,3-benzothiazol-3(2H)-yl)ethoxy]phenyl}propanoate,
methyl 2-[(tert-butoxycarbonyl)(methyl)amino]-3-{4-[2-(6-[(methoxyimino)(phenyl)-methyl]-2-oxo-1,3-benzothiazol-3(2H)-yl)ethoxy]phenyl}propanoate,
N-(tert-butoxycarbonyl)-4-[2-(6-[(hydroxyimino)(phenyl)methyl]-2-oxo-1,3-benzothiazol-3(2H)-yl)ethoxy]phenylalanine,
N-(tert-butoxycarbonyl)-4-[2-(6-[(methoxyimino)(phenyl)methyl]-2-oxo-1,3-benzothiazol-3(2H)-yl)ethoxy]phenylalanine,
methyl 2-amino-3-{4-[2-(6-[(methoxyimino)(phenyl)methyl]-2-oxo-1,3-benzothiazol-3(2H)-yl)ethoxy]phenyl}propanoate,
methyl 2-amino-3-{4-[2-(6-[(hydroxyimino)(phenyl)methyl]-2-oxo-1,3-benzothiazol-3(2H)-yl)ethoxy]phenyl}propanoate,
methyl 3-{4-[2-(6-[(methoxyimino)(phenyl)methyl]-2-oxo-1,3-benzothiazol-3(2H)-yl)ethoxy]phenyl}-2-(methylamino)propanoate,
   their enantiomers and diastereoisomers, and also addition salts thereof with a pharmaceutically acceptable acid or base.

Antioxidant agents according to the invention are, more specifically, anti-free radical agents or free-radical trapping agents, antilipoperoxidant agents, chelating agents or agents capable of regenerating endogenous antioxidants such as glutathione, vitamin C or vitamin E, and also addition salts thereof with a pharmaceutically acceptable acid or base.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, etc.

Amongst the pharmaceutically acceptable bases there may be mentioned, without implying any limitation, sodium hydroxide, potassium hydroxide, triethylamine, tert-butylamine, etc.

The antioxidant agent of the association according to the invention is more preferably represented by quinone compounds such as ubiquinone or coenzyme Q₁₀, which acts as a free-radical trapping agent but which is also capable of regenerating vitamin E.

The association to which preference is given in accordance with the invention is 2-ethoxy-3-{4-[2-(6-[(hydroxyimino)(phenyl)methyl]-2-oxo-1,3-benzothiazol-3(2H)-yl)ethoxy]-phenyl}propanoic acid and coenzyme Q₁₀.

Furthermore, the association according to the invention between a compound favouring the lipid and carbohydrate metabolisms and an antioxidant agent has entirely surprising pharmacological properties: the Applicant has discovered that a synergy exists between the two compounds of the association allowing a very significant reduction in body fat to be obtained, making it of use in the treatment and/or prevention of obesity and of overweight characterised by a body mass index greater than 25.

In the United States, obesity affects 20% of men and 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426). Obesity (BMI≧30) and overweight (25<BMI<30) can have various origins: they may come about following deregulation of food intake, following hormonal disturbance, or following administration of a treatment: treating type II diabetes with sulphonylureas causes patients to gain weight. Similarly, in type I (insulin-dependent) diabetes, insulin therapy is also a cause of weight gain in patients (In Progress in Obesity Research, 8^{th} International Congress on Obesity, 1999, 739-746; Annals of Internal Medicine, 1998, 128, 165-175).

Obesity and overweight are well-established risk factors for cardiovascular diseases: they are associated with a significant increase in the risk of cerebro-vascular accidents and non-insulin-dependent diabetes, because they predispose to insulin-resistance, dyslipidaemia and the appearance of macrovascular disorders (nephropathy, retinopathy, angiopathy). Further pathologies are the consequence of obesity or overweight: there may be mentioned, in particular, vesicular calculi, respiratory dysfunction, several forms of cancer and, in the case of very severe obesity, premature death (N. Engl. J. Med., 1995, 333, 677-385; JAMA, 1993, 270, 2207-2212).

The association according to the invention allows a weight loss to be obtained which, even if moderate, significantly reduces all the risk factors associated with obesity (Int. J. Obes., 1997, 21, 55-9; Int. J. Obes., 1992, 21, S5-9).

The association according to the invention will therefore be found to be useful in the treatment and/or prevention of obesity and of overweight characterised by a body mass index greater than 25.

The invention accordingly relates to the use of the association between a compound favouring the lipid and carbohydrate metabolisms and an antioxidant agent in obtaining pharmaceutical compositions intended for the treatment and/or prevention of obesity and of overweight characterised by a body mass index greater than 25 and less than 30.

In particular, the association according to the invention is of use in the treatment and/or prevention of obesity and of overweight characterised by a body mass index greater than 25 and less than 30 caused by a therapeutic treatment, such as treatment for type I or II diabetes.

The invention accordingly relates to the use of the association between a compound favouring the lipid and carbohydrate metabolisms and an antioxidant agent in obtaining pharmaceutical compositions intended for the treatment and/or prevention of obesity and of overweight characterised by a body mass index greater than 25 and less than 30 caused by a therapeutic treatment, such as treatment for type I or II diabetes.

The invention relates also to pharmaceutical compositions comprising the association between a compound favouring the lipid and carbohydrate metabolisms and an antioxidant agent, as defined hereinbefore, in combination with one or more pharmaceutically acceptable excipients.

Among the pharmaceutical compositions according to the invention, there may be mentioned, more especially, those that are suitable for oral, parenteral or nasal administration, tablets or dragées, sublingual tablets, gelatin capsules, lozenges, suppositories, creams, ointments, dermal gels, etc.

In particular, the invention relates to pharmaceutical compositions comprising a compound of formula (I) as defined hereinbefore and an antioxidant agent such as coenzyme Q₁₀ or vitamin E, in combination with one or more pharmaceutically acceptable excipients. The dosage used varies according to the sex, age and weight of the patient, the administration route, the nature of the therapeutic indication or of any associated treatments and ranges from 0.1 mg to 1 g of each component of the association per 24 hours in one or more administrations.

The following Examples illustrate the invention but do not limit it in any way.

### EXAMPLE A

### Change in Body Weight

Male C57 Black 6 ob/ob mice from 8 to 12 weeks old were used. After being placed in quarantine for one week, they were weighed and then randomised as a function of their weight and 6 homogeneous groups (starting weights not significantly different) were formed. After being weighed, the various compounds under test were injected by the intraperitoneal route once a day for 7 days. The compounds were injected in a solution of DMSO 5%/Solutol 15%/qsp H₂O heated at 65° C. to ensure good dissolution. In addition, the solution was pre-heated before injection. The mice were weighed every day and the weight obtained after 7 days of treatment was recorded.

The results obtained with the association according to the invention show:
that the association enables the weight of obese mice to be reduced significantly,
that there exists a synergy between the 2 components of the association, the weight loss found being much greater in the case of the association than in the case of each component administered on its own.

In particular, a reduction of 30% is observed in the weight gain of mice treated with the association comprising 2-ethoxy-3-{4-[2-(6-[(hydroxyimino)(phenyl)methyl]-2-oxo-1,3-benzothiazol-3(2H)-yl)ethoxy]phenyl}propanoic acid and coenzyme Q₁₀, whereas coenzyme Q₁₀ administered on its own does not reduce the weight gain and 2-ethoxy-3-{4-[2-(6-[(hydroxyimino)(phenyl)methyl]-2-oxo-1,3-benzothiazol-3(2H)-yl)ethoxy]phenyl}-propanoic acid administered on its own reduces the weight gain by only 10%.

### EXAMPLE B

### Pharmaceutical Composition

100 tablets each containing 30 mg of 2-ethoxy-3-{4-[2-(6-[(hydroxyimino)(phenyl)-methyl]-2-oxo-1,3-benzothiazol-3(2H)-yl)ethoxy]phenyl}propanoic acid and 10 mg of coenzyme Q₁₀
2-ethoxy-3-{4-[2-(6-[(hydroxyimino)(phenyl)methyl]-2-oxo-1,3-benzo-thiazol-3(2H)-yl)ethoxy]phenyl}propanoic acid . . . 3 g
coenzyme Q₁₀ . . . 1 g
wheat starch . . . 20 g
maize starch . . . 20 g
lactose . . . 30 g
magnesium stearate . . . 2 g
silica . . . 1 g
hydroxypropylcellulose . . . 2 g

1-16. (canceled)

17. A pharmaceutical composition for treatment of obesity associated with lipid and carbohydrate metabolism comprising (i) a substance which is useful in promoting lipid and carbohydrate metabolism, (ii) an antioxidant agent and, optionally, (iii) a pharmaceutically acceptable carrier or excipient, wherein the substance which promotes lipid and carbohydrate metabolism and the antioxidant agent are present in therapeutically effective dosages.

18. The composition of claim 17, wherein the substance which promotes lipid and carbohydrate metabolism is a compound selected from those of formula (1): 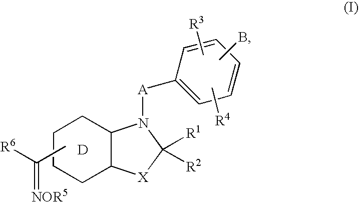
wherein
X represents an oxygen or sulphur atom, or a group CH₂ or 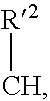
wherein R′² together with R² forms an additional bond,
R¹ and R², which may be the same or different, each represent a hydrogen atom, a linear or branched (C₁-C₆)alkyl group, an aryl group, an aryl-(C₁-C₆)alkyl group in which the alkyl moiety is linear or branched, an aryloxy group, an aryl-(C₁-C₆)alkyloxy group in which the alkyl moiety is linear or branched, a linear or branched (C₁-C₆)alkoxy group, a hydroxy group, an amino group, a linear or branched (C₁-C₆)alkylamino group or a di-(C₁-C₆)alkylamino group in which the alkyl moieties are linear or branched,
or R¹ and R² together form an oxo, thioxo or imino group, it also being possible for R² together with R′² to form an additional bond,
A represents a (C₁-C₆)alkylene chain in which one CH₂ group may be replaced by a hetero atom selected from oxygen and sulphur or by a group NRₐ, wherein Rₐ represents a hydrogen atom or a linear or branched (C₁-C₆)alkyl group, or by a phenylene or naphthylene group,
R³ and R⁴, which may be the same or different, each represent a hydrogen or halogen atom or a group R, OR or NRR′, wherein R and R′, which may be the same or different, each represent a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety is linear or branched, an aryl-(C₂-C₆)alkenyl group in which the alkenyl moiety is linear or branched, an aryl-(C₂-C₆)alkynyl group in which the alkynyl moiety is linear or branched, a heteroaryl group, a heteroaryl-(C₁-C₆)alkyl group in which the alkyl moiety is linear or branched, a heteroaryl-(C₂-C₆)alkenyl group in which the alkenyl moiety is linear or branched, a heteroaryl-(C₂-C₆)alkynyl group in which the alkynyl moiety is linear or branched, a (C₃-C8)cycloalkyl group, a (C₃-C₈)cycloalkyl-(C₁-C₆)alkyl group in which the alkyl moiety is linear or branched, or a linear or branched (C₁-C6)polyhaloalkyl group,
or R³ and R⁴, together with the carbon atoms carrying them, when they are carried by two adjacent carbon atoms, form a ring that has 5 or 6 ring members and that may contain a hetero atom selected from oxygen, sulphur and nitrogen,
R⁵ and R⁶, which may be the same or different, each represent a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety is linear or branched, an aryl-(C₂-C₆)alkenyl group in which the alkenyl moiety is linear or branched, an aryl-(C₂-C₆)alkynyl group in which the alkynyl moiety is linear or branched, a heteroaryl group, a heteroaryl-(C₁-C₆)alkyl group in which the alkyl moiety is linear or branched, a heteroaryl-(C₂-C₆)alkenyl group in which the alkenyl moiety is linear or branched, a heteroaryl-(C₂-C₆)alkynyl group in which the alkynyl moiety is linear or branched, a (C₃-C₈)cycloalkyl group, a (C₃-C₈)cycloalkyl-(C₁-C₆)alkyl group in which the alkyl moiety is linear or branched, or a linear or branched (C₁-C₆)polyhaloalkyl group,
D represents:
a benzene nucleus, in which case X cannot represent a group 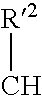
or D represents a pyridine, pyrazine, pyrimidine or pyridazine nucleus,
B represents a linear or branched (C₁-C₆)alkyl group or a linear or branched (C₂-C₆)-alkenyl group, those groups being substituted:
by a group of formula (II): 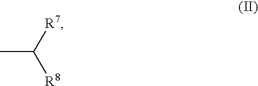
wherein:
R⁷ represents a group 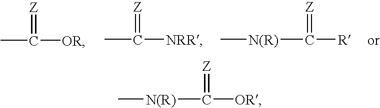
wherein Z represents an oxygen or sulphur atom,
and R⁸ represents an aryl group, an arylalkyl group wherein the alkyl moiety contains from 1 to 6 carbon atoms and may be linear or branched, a heteroaryl group, a heteroarylalkyl group wherein the alkyl moiety contains from 1 to 6 carbon atoms and may be linear or branched, CN, tetrazole, 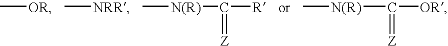
or by a group R⁹, wherein R⁹ represents a CN, tetrazole, 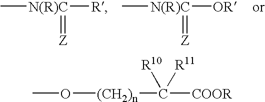
group, wherein n represents 0, 1, 2, 3, 4, 5 or 6, and R¹⁰ to and R¹¹, which may be the same or different, each represent a hydrogen atom or a linear or branched (C₁-C₆)alkyl group, it being understood that R₁₀ and R₁₁ cannot simultaneously represent a hydrogen atom,
or B represents a group of formula (II) or a group R⁹,
it being understood that:
the oxime R⁶—C(═N—OR⁵)- can be of Z or E configuration,
aryl means a phenyl, naphthyl or biphenyl group, it being possible for those groups to be partially hydrogenated,
heteroaryl means any mono- or bi-cyclic aromatic group containing 5 to 10 members, which may be partially hydrogenated in one of the rings in the case of bicyclic heteroaryls and which contains 1 to 3 hetero atoms selected from oxygen, nitrogen and sulphur,
wherein the aryl and heteroaryl groups may be optionally substituted by from 1 to 3 groups selected from linear or branched (C₁-C₆)alkyl, linear or branched (C₁-C₆)polyhaloalkyl, linear or branched (C₁-C₆)alkoxy, hydroxy, carboxy, formyl, NR_{b}R_{c}, wherein R_{b} and R_{c}, which may be the same or different, each represent a hydrogen atom, a linear or branched (C₁-C₆)alkyl group, an aryl group or a heteroaryl group, ester, amido, nitro, cyano, and halogen atoms,
its enantiomers and diastereoisomers thereof, and addition salts thereof with a pharmaceutically acceptable acid or base.

19. The composition of claim 1, wherein the substance which promotes lipid and carbohydrate metabolism is 2-ethoxy-3-{4-[2-(6-[(hydroxyimino)(phenyl)methyl]-2-oxo-1,3-benzothiazol-3(2H)-yl)ethoxy]phenyl}propanoic acid, its enantiomers and diastereoisomers thereof, and addition salts thereof with a pharmaceutically acceptable acid or base.

20. The composition of claim 17, wherein the antioxidant agent is coenzyme Q₁₀.

21. The composition of claim 17, wherein the antioxidant agent is vitamin E.

22. The composition of claim 17, which is 2-ethoxy-3-{4-[2-(6-[(hydroxyimino)(phenyl)methyl]-2-oxo-1,3-benzothiazol-3(2H)-yl)ethoxy]phenyl}propanoic acid and coenzyme Q₁₀.

23. The composition of claim 17, which is 2-ethoxy-3-{4-[2-(6-[(hydroxyimino)(phenyl)methyl]-2-oxo-1,3-benzothiazol-3(2H)-yl)ethoxy]phenyl}propanoic acid and vitamin E.

24. A method for treating a living animal body, including a human, afflicted with obesity, comprising the step of administering to the living animal body, including a human, an amount of a composition of claim 17 which is effective for alleviation of obesity.

25. A method for treating a living animal body, including a human, afflicted with obesity caused by a therapeutic treatment, comprising the step of administering to the living animal body, including a human, an amount of a composition of claim 17 which is effective for alleviation of obesity caused by a therapeutic treatment.

26. A method for treating a living animal body, including a human, afflicted with obesity caused by treatment for type I or II diabetes, comprising the step of administering to the living animal body, including a human, an amount of a composition of claim 17 which is effective for alleviation of obesity caused by treatment for type I or II diabetes.

27. A method for treating a living animal body, including a human, afflicted with overweight characterised by a body mass index greater than 25 and less than 30, comprising the step of administering to the living animal body, including a human, an amount of a composition of claim 17 which is effective for alleviation of overweight characterised by a body mass index greater than 25 and less than 30.

28. A method for treating a living animal body, including a human, afflicted with overweight characterised by a body mass index greater than 25 and less than 30 caused by a therapeutic treatment, comprising the step of administering to the living animal body, including a human, an amount of a composition of claim 17 which is effective for alleviation of overweight characterised by a body mass index greater than 25 and less than 30 caused by a therapeutic treatment.

29. A method for treating a living animal body, including a human, afflicted with overweight characterised by a body mass index greater than 25 and less than 30 caused by treatment for type I or II, diabetes comprising the step of administering to the living animal body, including a human, an amount of a composition of claim 17 which is effective for alleviation of overweight characterised by a body mass index greater than 25 and less than 30 caused by treatment for type I or II diabetes.
